(19) United States
(12) Patent Application Publication
(10) Pub. No.: US 2018/0008122 A1
(43) Pub. Date: Jan. 11, 2018

(54) CATHETER TUBE

(71) Applicant: Arai Medphoton Research Laboratories, Corporation, Kanagawa (JP)

(72) Inventors: Tsunenori ARAI, Kanagawa (JP); Emiyu OGAWA, Kanagawa (JP)

(21) Appl. No.: 15/643,870

(22) Filed: Jul. 7, 2017

(30) Foreign Application Priority Data
Jul. 8, 2016 (JP) 2016-136229

(51) Int. Cl. A61B 1/00; A61M 25/00; A61B 1/06

(52) U.S. Cl. CPC A61B 1/00013; A61M 25/0015; A61M 25/0014; A61B 1/0661; A61M 25/0043

(57) ABSTRACT

Catheter tube capable of irradiating laser optimized in light radiation efficiency and distribution, used in photosensitization reaction treatment is provided by simple process. Catheter tubes provided to medical device inserted into a living body to irradiate target site for treatment or examination with a light to treat or examine the site. The tubes being formed of material optically having transparency, provided with insertion hole for light diffusing body, and one or more other holes different from the insertion hole, in a manner extending in longitudinal direction of catheter tubes. Inside the other holes, space is present adjacent to inner wall thereof, the inner wall forming optical interface between the material and gas phase or liquid phase in the space, which are different from each other in refractive index, to conjointly form reflection surface for adjusting radiation distribution of light emitted by light diffusing body.

### CROSS REFERENCE TO RELATED APPLICATIONS

This application is based on and claims the benefit of priority from the prior Japanese Patent Application No. 2016-136229, filed on Jul. 8, 2016, the entire contents of which are incorporated herein by reference.

### BACKGROUND OF THE INVENTION

### Field of the Invention

The present invention relates to a catheter tube which is used by being inserted into a living body, comprising a light diffusing body for irradiating a target site for treatment or examination with light.

### Description of the Related Art

Recent years, in treatments of cancers or tachyarrhythmias, a tube internally comprising a light diffusing body, which is designed to have a plastic optical fiber with a cladding layer polished to radiate a diffused light from a side face, is utilized in treatments which use a light, such as photodynamic treatments.

Here, the photodynamic treatment refers to a method of treating a target tissue by administering a photosensitive substance to the target tissue by means of intravenous injection or the like, and irradiating the target tissue in a condition of having the photosensitive substance distributed with a light such as a laser light, to generate a photosensitization reaction by the photosensitive substance, the light and oxygen, to necrose cells of a target tissue by the photosensitization reaction.

In a photodynamic treatment, principal properties which a design of the tube is required to have are two points which are radiation efficiency and radiation distribution of light. The light radiation efficiency is an important property, because a tube having a low radiation efficiency cannot provide an effective treatment energy, and an energy loss causes a heat, and it requires measures for preventing a tissue or blood lesion due to the heat.

In addition, in photodynamic treatments, measures for optimizing the light radiation distribution is desired, because while irradiation of a target tissue with a light such as a laser light necroses cells of the target tissue, there is a need of inhibiting an irradiation of blood or tissues other than target tissues.

Therefore, a laser catheter used in photodynamic treatments of a tachyarrhythmias which is provided with a reflection layer for adjusting the radiation efficiency and radiation distribution has been suggested (For example, Japanese National-Phase Publication 2012-510084 (JP 2012-510084 A).

A laser catheter 101 of JP 2012-510084 A is a laser catheter in a shape sometimes called as lasso-type that has a circular distal end loop-like shaped, which is provided with a reflection layer.

FIG. 15 shows a cross section of a loop-like shaped portion of a distal end where a laser energy is output from the catheter in a direction of the side face, in the laser catheter 101 of JP 2012-510084 A. The loop-like shaped portion of the laser catheter 101 comprises a lumen 118 into which a shape memory wire 119 for maintaining a loop shape is inserted, two longitudinal cooling channels 120 and 121, and an optical fiber 126.

A substantially v-shaped groove 116 which is covered with a reflection layer 117 is formed so as to surround the optical fiber 126, and the optical fiber 126 is fixed inside the groove 116 with an adhesive 128. A light emitted from the optical fiber 126 is radiated only in an outward direction of the loop shape.

By configuring in this manner, it is possible to radiate a light only to a site in need of a treatment, in the radial outward direction of the loop shape, with preventing the light from being radiated to blood, etc. in the radial direction toward the center, to thus prevent a temperature rise, etc. of blood, etc.

However, in the laser catheter of JP 2012-510084 A, the production steps of the laser catheter were complicated, and at the same time, number of the production steps was large, because, after the catheter comprising the groove 116 was produced, it is necessary to form the reflection layer 117 by a metal plating or the like, on a surface of the groove 116, and further, to fix the optical fiber 126 inside the groove 116.

Moreover, since a metal plating process or an adhesion process with an adhesive within a laser catheter was a minute work, it was hard to process a small diameter laser catheter, in an increased demand for a laser catheter with a smaller diameter.

In addition, as described above, principal properties required in designing a catheter tube are the two points which were radiation efficiency and radiation distribution, and the radiation distribution needs to be designed according to a treatment site.

For example, in a treatment of arrhythmia by a photosensitization reaction, major targets for treatment are pulmonary vein and superior vena cava. In a light irradiation of a pulmonary vein, a portion a little forward from the outer side of a ring shaped catheter tube is brought into contact with a tissue. Therefore, a light irradiation to a wide area is required in order to secure a sufficient radiation exposure dose.

On the other hand, in a light irradiation of a superior vena cava, it is possible that an outer side of a ring shaped catheter tube is brought into contact with a tissue. Therefore, there is no need of light irradiation to a wide area, and at the same time, there is a need of a light irradiation to a local area, since there is a very important site which is called sinoatrial node, in the vicinity of a site to be irradiated with light.

However, such a tube for a laser catheter having a radiation distribution designed according to a treatment site has not been conventionally known.

Moreover, a catheter tube internally comprising a light diffusing body for cancer treatment which has an improved radiation efficiency and an improved radiation distribution has not been known.

The present invention has been made in view of the above issues, and object of the present invention is to provide, by a simple process, a catheter tube capable of irradiating a laser with an optimized light radiation efficiency and an optimized light distribution, which is used in a treatment by a photosensitization reaction.

Further object of the present invention is to provide a catheter tube capable of irradiating a laser with an optimized light radiation efficiency and an optimized light distribution, which is used in a treatment by a photosensitization reaction, capable of being used not only in an arrhythmia treatment, but also in a cancer treatment by a photosensitization reaction.

### SUMMARY OF THE INVENTION

According to the catheter tube of the present invention, the problems are solved by a catheter tube provided to a medical device which is inserted into a living body to irradiate a target site for treatment or a target site for examination with a light to treat or examine the site, which is formed with a material optically having an transparency, and comprises an insertion hole for light diffusing body through which a light diffusing body is inserted, and one or more other holes which are different from the insertion hole for light diffusing body, in a manner extending in a longitudinal direction of the catheter tube, wherein a space presents inside the other holes, adjacent to an inner wall face of the other hole, such that the inner wall face forms an optical interface between the material and a gas phase or a liquid phase inside the space which are different from each other in refractive index, to conjointly form a reflection face which adjusts a radiation distribution of a light emitted by the light diffusing body.

As configured in this manner, it becomes possible to freely adjust a radiation distribution and a radiation efficiency of a light from a catheter tube which comprises a light diffusing body, with a simple configuration.

Since the inner wall face of the one or more other holes which are different from the insertion hole for light diffusing body forms an optical interface between the material and a gas phase inside the space which are different from each other in refractive index, to conjointly form a reflection face which adjusts a radiation distribution of a light emitted by the light diffusing body, there is no need of specially installing a reflection body such as a mirror or a prism in the catheter tube, and thus, the catheter tube is inhibited from becoming stiff, and it is possible to realize a flexible structure with a small diameter. As a result, it is possible to obtain a catheter tube having a favorable maneuverability in a living body, and an optimized light radiation distribution.

In addition, it is possible to easily adjust a light radiation distribution of a catheter tube, by only changing a setting of a conventional extrusion molding device, since it is possible to adjust a light radiation distribution by adjusting arrangement of the other holes. As a result, it is possible to easily respond to demands for suitable radiation distribution which vary depending on a target site for a treatment or an examination, allowing a treatment or an examination of a target site which has been conventionally difficult, and it becomes possible to improve an accuracy of a treatment or an examination.

Moreover, the catheter tube may not be provided with a reflection body which comprises a material different from that of a catheter tube.

Furthermore, the catheter tube may be mirrorless or prismless, where a mirror or a prism for adjusting a radiation distribution, comprising a material different from that of the catheter tube, is not provided.

By configuring in this manner, it becomes possible to configure a catheter tube without a reflection body, a mirror, a prism, or the like which is made of a different material, and thus, it becomes possible to simplify manufacturing steps of a catheter tube. Moreover, it is possible to make a catheter tube having further smaller diameter, since the substance of different material can be omitted. Furthermore, since it is possible to inhibit a catheter tube from becoming stiff due to the substance of different material, it is possible to obtain a flexible catheter tube with a high maneuverability in a living body or in a channel of an endoscope.

Further, the catheter tube may be one for treatment of arrhythmia which has: a loop-like shaped portion formed with a spiral shape of more than a single convolution under a condition without an external stress; and a tip portion comprising a bent portion which is bent at the end portion in the proximal side of the loop-like shaped portion.

By configuring into loop-like shape, a catheter tube is allowed to contact with a pulmonary vein or a superior vena cava which is a principal genesis location of a tachyarrhythmia, and to perform a light irradiation at a time, and thus, it is possible to provide a catheter tube capable of drawing a blocking line at a time by a single time light irradiation.

The catheter tube may be a wide angle irradiation catheter tube for treatment of tachyarrhythmia at an inlet portion of a pulmonary vein or treatment of peripheral lung cancer by a photosensitization reaction, which comprises first to fourth holes provided in the side of the center axis of the catheter tube rather than the center axis of the insertion hole for light diffusing body as the other holes: the first hole being formed in a position substantially coaxial with the catheter tube; the second hole being formed in the opposite side of the first hole from the insertion hole for light diffusing body; such that each center axis of the insertion hole for light diffusing body, the first hole, and the second hole are arranged in a straight line which passes through the center axis of the insertion hole for light diffusing body and the center axis of the catheter tube; and such that center axes of the third and the fourth holes are positioned between the center axis of the insertion hole for light diffusing body and the center axis of the first hole, in the linear direction.

As configured in this manner, it is possible to produce a catheter tube having a wide light irradiation angle, with a simple configuration, and it is possible to provide a catheter tube suitable for a treatment of tachyarrhythmia and a treatment of peripheral lung cancer in an inlet portion of a pulmonary vein which are pathologies where a wide angle light radiation is desirable.

The catheter tube may be a narrow angle irradiation catheter tube for treatment of tachyarrhythmia in superior vena cava or treatment of central lung cancer, treatment of brainstem tumor or treatment of residual tumor after surgical operation by a photosensitization reaction, which has first to fourth holes as the other holes, provided in the side of the center axis of the catheter tube rather than the center axis of the insertion hole for light diffusing body: the first hole being formed in a position substantially coaxial with the catheter tube; the second hole being formed in the opposite side of the first hole from the insertion hole for light diffusing body; such that each center axis of the insertion hole for light diffusing body, the first hole, and the second hole are arranged in a straight line which passes through the center axis of the insertion hole for light diffusing body and the center axis of the catheter tube; and such that the third and the fourth holes are positioned between the center axis of the second hole and the center axis of the first hole, in the linear direction.

As configured in this manner, it is possible to produce a catheter tube having a narrowed light irradiation angle, with a simple configuration, and it is possible to provide a catheter tube suitable for a treatment of tachyarrhythmia in superior vena cava or a treatment of central lung cancer, a treatment of brainstem tumor or a treatment of residual tumor after surgical operation by a photosensitization reaction which are pathologies where a narrow angle light radiation is desirable.

In particular, in treatment of brainstem tumor or treatment of residual tumor after surgical operation, where there has conventionally been no alternative treatment method, a remarkable treatment has been allowed by a complementary use of the catheter tube of the present invention in combination with other treatment method such as a surgical treatment.

According to the method of conducting a treatment or an examination of the present invention, the problems are solved by a method of inserting a catheter tube provided to a medical device into a body of a patient, and irradiating a target site for treatment or a target site for examination with a light by using the catheter tube, to conduct a treatment or an examination of a cancer or a arrhythmia in the site, in which the catheter tube comprises a material optically having a transparency; the catheter tube is provided with an insertion hole for light diffusing body through which a light diffusing body is inserted, and one or more other holes different from the insertion hole for light diffusing body, in a manner extending in a longitudinal direction of the catheter tube; a space presents inside the other hole, adjacent to an inner wall face of the other hole; and the inner wall face forms an optical interface between the material and a gas phase or a liquid phase inside the space which are different from each other in refractive index, to conjointly form a reflection face which adjusts a radiation distribution of a light emitted by the light diffusing body.

### (Effect)

According to the present invention, it becomes possible to adjust a radiation distribution and a radiation efficiency of a light from a catheter tube which comprises a light diffusing body, with a simple configuration.

Since the inner wall face of the one or more other holes which are different from the insertion hole for light diffusing body forms an optical interface between the material and a gas phase inside the space which are different from each other in refractive index, to conjointly form a reflection face which adjusts a radiation distribution of a light emitted by the light diffusing body, there is no need of specially installing a reflection body such as a mirror or a prism in the catheter tube, and thus, it is possible to inhibit the catheter tube from becoming stiff. As a result, it is possible to obtain a catheter tube having a favorable maneuverability in a living body, and an optimized light radiation distribution.

In addition, it is possible to easily adjust a light radiation distribution of a catheter tube, by only changing a setting of a conventional extrusion molding device, since it is possible to adjust a light radiation distribution by adjusting arrangement of the other holes. As a result, it is possible to easily respond to demands for suitable radiation distribution which vary depending on a target site for a treatment or an examination, allowing a treatment or an examination of a target site which has been conventionally difficult, and it becomes possible to improve an accuracy of a treatment or an examination.

### BRIEF DESCRIPTION OF THE DRAWINGS

FIG. 1 is an exterior explanatory view of a catheter tube according to an embodiment of the present invention.

FIG. 2 is a schematic explanatory view showing a structure of a general pulmonary vein.

FIG. 3 is a cross-sectional explanatory view of the catheter tube according to Embodiment 1 of the present invention at an A-A cross-sectional view of FIG. 1.

FIG. 4 is a cross-sectional explanatory view of the catheter tube according to Embodiment 2 of the present invention, at a part corresponding to the A-A cross-section of FIG. 1.

FIG. 5 is a cross-sectional explanatory view of the catheter tube according to Embodiment 3 of the present invention, at a part corresponding to the A-A cross-section of FIG. 1.

FIG. 6 is a cross-sectional explanatory view of the catheter tube according to Embodiment 4 of the present invention, at a part corresponding to the A-A cross-section of FIG. 1.

FIG. 7 shows cross-sectional explanatory views of the catheter tubes according to other modifications of the present invention, at a part corresponding to the A-A cross-section of FIG. 1.

FIG. 8 shows cross-sections of 4 lumen-catheter tube (a) and 5 lumen-catheter tube (b); and a radiation distribution (c) of the 4 lumen-catheter tube and a radiation distribution (d) of the 5 lumen-catheter tube.

FIG. 9 is an explanatory view showing an experimental system for measuring a radiation distribution from a catheter tube internally comprising a light diffusing body.

FIG. 10 is an explanatory view concerning a study of arrangement of a lumen for electrode wire.

FIG. 11 is a graph showing a result of a study concerning relationship between an arrangement of lumen for electrode wire and a radiation intensity, a radiation angle, and a radiation efficiency.

FIG. 12 is a view showing radiation distributions from the catheter tubes according to Embodiments 2 and 4 of the present invention.

FIG. 13 is a view showing radiation intensity distributions inside a tissue when irradiated with a light from the catheter tube according to Embodiment 2 or 4 of the present invention.

FIG. 14 shows cross-sectional explanatory views of the catheter tubes according to other modification examples of the embodiment of the present invention, at parts corresponding to A-A cross-section of FIG. 1, with corresponding calculation results of radiation distributions.

FIG. 15 is an explanatory view showing a cross-sectional view of a loop-like shaped portion of a laser catheter tube according to a conventional example.

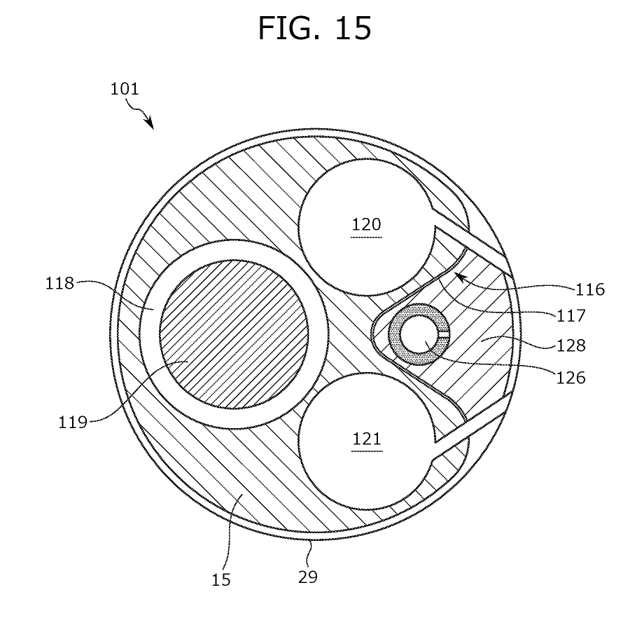

### DETAILED DESCRIPTION

The present embodiment describes a laser catheter 1 and a mode of being inserted into a channel of an endoscope, as an example of the catheter tube. However, the catheter tube of the present invention is not limited to those, but may only be a catheter tube to be introduced into a living body to a target tissue for a treatment, such as sheaths, vein conduits, artery conduits, bronchoscopes, cystoscopes, culpascopes, colonoscopes, trocars, laparoscopes, or other medical tubes.

Besides, the embodiment of the present invention describes examples of using the catheter tube of the present invention in a cancer treatment by a photodynamic treatment by a photosensitization reaction, and an arrhythmia treatment in which a line for blocking an abnormal electrical conduction is produced. However, the use of the catheter tube of the present invention is not limited to those. The catheter tube of the present invention may be used, for example, in a photodynamic treatment of an infectious disease or arteriosclerosis, or in a treatment of thrombosis in which a laser catheter is used. The catheter tube of the present invention may also be used in various treatments such as an endoscopic photodynamic treatment of cancer in a pancreas or in a biliary tract in which an endoscope of very small diameter, such as biliary tract endoscope (outer diameter: 1-3 mm) or pancreas endoscope (outer diameter: 1-2.5 mm) is used.

The catheter tube of the present invention may be used in any pathology where a medical device comprising a light irradiation probe represented by the laser catheter 1 or an endoscope can be used, and may be used in any case of conducting a laser irradiation or a laser measurement.

In the present specification, a proximal side of catheter tube refers to the outer side of a living body in a condition that a catheter tube is inserted into the living body, namely, the side of an operator; and a distal side of catheter tube refers to the tip side of the portion which is inserted into the living body, namely, the side of a target tissue for a treatment.

### Embodiment 1: Catheter Tube 20 Used in Treatment of Arrhythmia

Hereinbelow, a catheter tube 20 and a laser catheter 1 to which the catheter tube 20 is applied, according to an embodiment of the present invention are described.

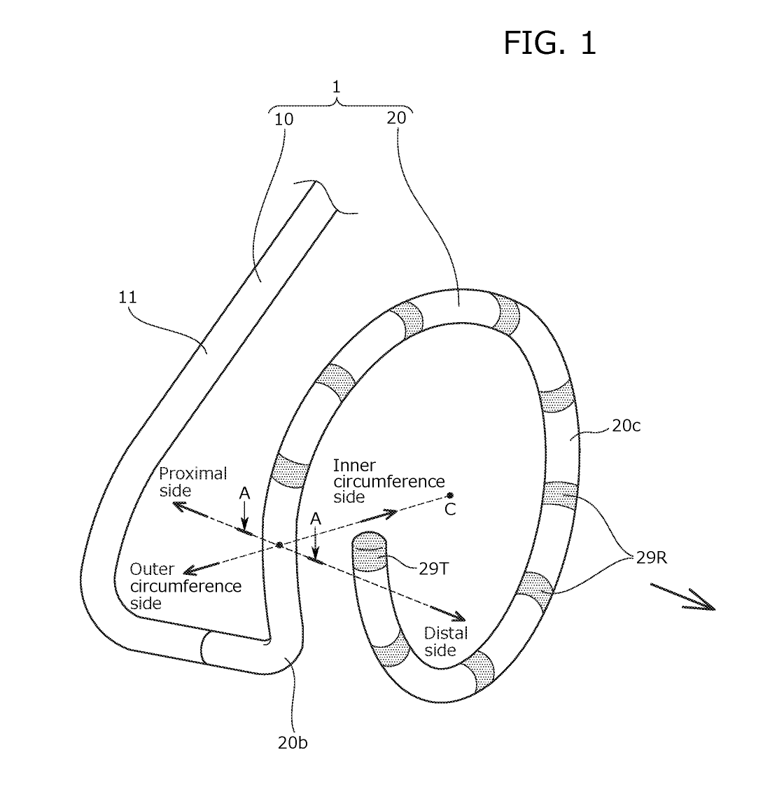

The laser catheter 1 of the present embodiment comprises a catheter tube 20 provided at the distal end of the laser catheter 1, and a long tubular portion 10 linked to the catheter tube 20 and proximally provided to the catheter tube 20, and a publicly known control handle (not illustrated) linked to a proximal portion in the opposite side from the catheter tube 20 of the tubular portion 10, as shown in FIG. 1. To a proximal portion of the control handle in the opposite side from the tubular portion 10, a publicly known connector (not illustrated) is provided. Through this connector, the control handle is connected to a publicly known control device (not illustrated) which controls laser radiation of excitation light from the catheter tube 20, or measurement of returned excitation light and fluorescence emitted by a PDT agent irradiated with the excitation light.

The laser catheter 1 of the present embodiment is preferably used by being connected to a control device (not illustrated) which implements an extra-cellular photodynamic treatment (Extra-cellular PDT) which is conducted with a sufficient amount of photosensitive substance (hereinbelow, PDT agent) distributed in extra-cellular stroma and inside blood vessels in target tissues for treatment.

The extra-cellular photodynamic treatment of the present embodiment is implemented under a condition that a PDT agent is distributed exterior of a living body, namely, in an extracellular fluid and/or a transcellular fluid, and at the same time, the PDT agent is continuously supplied due to vasopermeability.

In the present embodiment, the laser catheter 1 is used in a treatment of arrhythmia. However, the laser catheter 1 may be used in any extra-cellular photodynamic treatment or examination which is conducted with a sufficient amount of PDT agent distributed in extra-cellular stroma and inside blood vessels in target tissues for treatment, and may also be used in other treatment or examination, such as a photodynamic treatment of an infectious disease. The laser catheter 1 may also be used in a cancer treatment (including gastroenterology, respiratory surgery, brain surgery, dermatology, obstetrics and gynecology and ophthalmology), a treatment of arteriosclerosis in cardiology, a treatment of urethral disease or a treatment of prostate in urology, etc. where the PDT agent accumulates inside cells of target tissues for treatment, as long as a sufficient amount of the PDT agent and oxygen are continuously supplied to extra-cellular stroma and inside blood vessels in target tissues for treatment. The laser catheter 1 may also be used in angioplasty.

Among tachyarrhythmias, arrhythmias treated by using the laser catheter 1 of the present embodiment particularly includes all kinds of tachyarrhythmias ascribable to a presence of an abnormal electrical conduction site or a hyperexcitability generation site where radiofrequency ablation treatment has conventionally been conducted. Specifically, the laser catheter 1 may be used in AF (atrial fibrillation) including paroxysmal AF, persistent AF, permanent AF; AFL (atrial flutter); or paroxysmal supraventricular tachycardias including AVRT (atrioventricular reciprocating tachycardia), AVNRT (atrioventricular nodal reentrant tachycardia), and AT (atrial tachycardia).

The infectious disease includes MRSA infectious diseases, gingivitis, paradentitis, periimplantitis, herpes, stomatitis, inflammatory candidiasis, etc.

The PDT agent used in the present embodiment is a photosensitive substance.

Differently from a photodynamic treatment of a cancer, in the present embodiment, there is no need of accumulating an agent to tissues, and therefore, a Drug Light interval after an administration of agent to a light irradiation is set to a short time of about several minutes to several ten minutes, and a treatment is started in a short time after the administration of agent. Therefore, it is preferred to use a water-soluble photosensitive substance having a speedy excretion property, which is swiftly distributed in a stroma after an administration via intravenous injection or the like.

Specific examples may include ATX-S10 (670 nm) which is a chlorin-type agent having a chlorin skeleton (Iminochlorin aspartic acid derivative (Oriental Menthol Industry Ltd., right assigned to PHOTOCHEMICAL CO in 2000, Japanese Patent Application Laid-Open No. H06-80671 A); NPe6 (664 nm) (talaporfin sodium, Laserphyrin (registered trade mark), mono-L-aspartyl chlorin e6, Japanese Patent No. 2961074 B); mTHPC (652 nm); SnET2 (660 nm) (tin etiopurpurin, Miravant Medical Technologies); AlPcS (675 nm) (chloro aluminium sulphonated phthalocyanine), BPD-MA (690 nm) (benzoporphyrin derivative monoacid ring A, QLT Inc.); Lu-tex (732 nm) (Lutetium Texaphyrin); etc. Among them, the talaporfin sodium is preferred.

In a photodynamic treatment of an arrhythmia of the present embodiment, the photosensitization reaction is utilized in the treatment at an early stage after an administration of PDT agent, namely, at a timing where the PDT agent is distributed outside cells (stroma space), rather than inside cells.

First, when a PDT agent is administered for example, via an intravenous injection, the PDT agent is distributed in a stroma and blood vessel which are outside cells, at a high concentration. At this time, the outside cells are in a state of being continuously supplied with the PDT agent and oxygen always at a sufficient amount, via blood flow. The PDT agent is supplied due to permeability from a blood vessel to a stroma.

When a target site is irradiated with a laser light via catheter, with setting a Drug Light interval after an administration to a light irradiation to a short time of about several minutes to several hours, the PDT agent and oxygen are sufficiently supplied by a blood flow, and at the same time, an energy necessary to a reaction is supplied by a laser light, and a photosensitization reaction due to the PDT agent, light and oxygen occurs.

In the photosensitization reaction, PDT agent is excited by a light irradiation. Energy of this excited PDT agent is transferred to oxygen which is present outside cells, to generate active singlet oxygen (active oxygen).

A total production amount of the singlet oxygen is an irradiating time-integral value of an amount of singlet oxygen per unit time.

When excited singlet oxygen is produced, a destruction of a protein to which the PDT agent is bound, a conversion of the excited singlet oxygen into a triplet ground oxygen, and an agent bleaching take place, due to a strong oxidizing power of this singlet oxygen; and obtained as therapeutic effect are elimination of electro conductivity of myocardial cells due to oxidative lesions of ion channels and cell membranes, and an instant cell death such as cytoclasis (necrosis).

Here, the agent bleaching refers to a phenomenon that a photosensitive substance which is a PDT agent is destroyed by a singlet oxygen.

### <<Structure of Catheter Tube 20>>

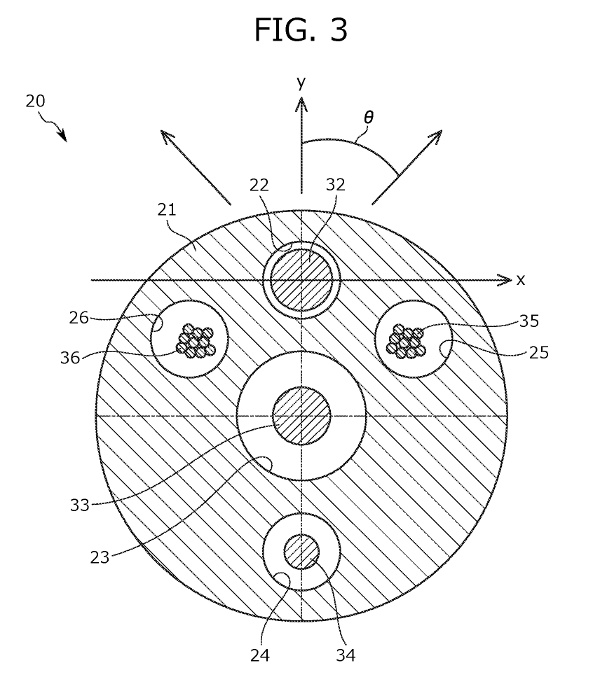

Hereinbelow, the laser catheter 1 and the catheter tube 20 according to Embodiment 1 are shown in FIGS. 1 and 3.

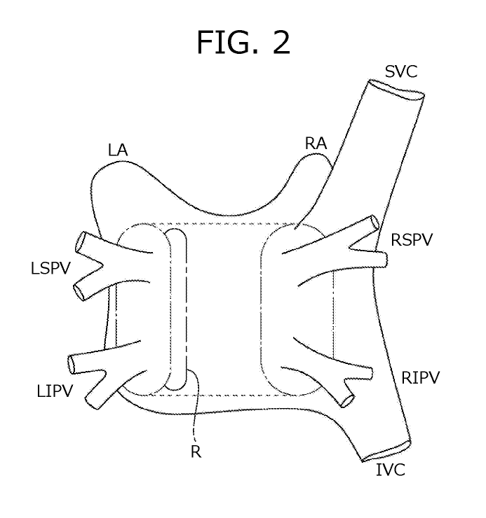

The catheter tube 20 of the present embodiment is, for example, a catheter tube which is capable of wide angle irradiation, and used in insulating an abnormal electrical conduction at an inlet portion of a pulmonary vein shown in FIG. 2, in a treatment of tachyarrhythmia.

As shown in FIG. 1, shape of catheter tube 20 is sometimes called as lasso-type, and is not strictly a closed ring shape, but is a quasi-ring shape having center C as the center, and accordingly, also called as ring-shaped or annular-shaped. The catheter tube 20 comprises a loop-like shaped portion 20c extending in a spiral from the distal tip portion 20t to the bent portion 20b, which is linked to the tubular portion 10 at the proximal side of the bent portion 20b.

FIG. 3 shows an A-A line sectional view of the catheter tube 20 of FIG. 1.

The catheter tube 20 comprises a transparent tube 21 which is formed by extrusion molding from a flexible transparent material by a publicly known method. The transparent tube 21 comprises five lumens consisting of long holes, namely, lumen 22 for light diffusing body, lumen 23 for shape memory wire, lumen 24 for tension wire, and lumens 25 and 26 for electrode wire, as shown in FIG. 3. These lumens 22 to 26 extend through the entire length of the catheter tube 20 from a vicinity of the tip portion 20t in the distal side of the catheter tube 20, and continue to lumens (not illustrated), individually formed inside the tubular portion 10.

The transparent tube 21 is a soft transparent tube having a hollow cylindrical shape, comprising polyether block amide copolymer (Pebax (registered trademark) manufactured by ARKEMA Co., Ltd) or the like. For example, Pebax (registered trademark) 55D may be used. In addition thereto, it is also possible to use an optically transparent and electrically nonconductive polymeric material, such as polyethylene terephthalate (PET), polyvinyl chloride (PVC), fluoroplastic (FEP) heat shrink tube, or the like, as the transparent tube 21.

Through the lumen 22 for light diffusing body, a light diffusing body 32 is inserted as shown in FIG. 3.

The light diffusing body 32 is configured with a light diffusing body (not illustrated) which is an optical fiber cable (not illustrated) inserted through the tubular portion 10, having a cladding and a coating removed therefrom to expose a core over a specific length. In this connection, it is also possible that the light diffusing body 32 is configured such that an outer circumference of a light diffusing body (not illustrated) having the core exposed is coated with a resin layer (not illustrated).

The light diffusing body 32 is configured to be integrated with the core of the optical fiber cable (not illustrated) inserted inside the tubular portion 10, and formed through a sand-blast process, so as to have a uniformized sideward radiation light at an angle with the longitudinal direction of the light diffusing body 32. Incidentally, the light diffusing body 32 is not limited thereto, but may also be provided with a hollow portion in the center, with the inner face thereof provided with a light reflection mirror, or with the inner face thereof notched, to uniformize the sideward radiation light. It is also possible to uniformize the sideward radiation light by providing an unevenness by a chemical process.

The resin layer (not illustrated) of the light diffusing body 32 is formed, for example, by applying a fine powder of quartz onto an acryl-based ultraviolet-curable resin, which is then cured with an ultraviolet light.

At the end portion of the optical fiber cable (not illustrated) in the tubular portion 10, in the opposite side from the light diffusing body 32, a connecter (not illustrated) is fixed, to configure the end portion linkable to a control device (not illustrated) which internally comprises a laser generation source (not illustrated).

Methods of configuring the light diffusing body 32 are roughly categorized into: a case where the core of the optical fiber cable (not illustrated) is extended to configure the light diffusing body 32; and a case where a light diffusing body 32 which is a body separate from the core is provided, and both light diffusing body may be used as the light diffusing body 32 of the present embodiment.

The former includes cases where the core constitutes/does not constitute the diffusing substance itself. Specifically, the former is roughly categorized into transmission light leakage systems (such as a system of creating small scratches in the cladding to expose part of the core, and a system of generating leakage by bending), and systems of using a diffusing substance.

The transmission light leakage systems include scratching processes (such as sand blasts, stampings, solvent treatments, etc.), Fiber Bragg Grating (FBG), microbendings, etc.

The systems of using a diffusing substance include systems of incorporating a diffusing substance into the core/cladding, systems of exposing the core and incorporating a diffusing substance inside the coating, etc. In this connection, sand blasts are categorized also into the system of using a diffusing substance, for being methods of blasting fine particles.

A case of using an optical element different from the core as the light diffusing body 32 corresponds to the latter which is the case where a light diffusing body 32 which is a body separate from the core is provided. Such case is, for example, a case of using an optical element, such as a polyhedric prism, SELFOC (registered trademark) lens (refractive index distribution-type lens), as the light diffusing body.

The light diffusing body 32 extends over the entire length of the catheter tube 20. When used in a photodynamic treatment of arrhythmia, a proper diameter of the spiral shape in FIG. 1 is from 5 to 50 mm, preferably from 10 to 30 mm. Accordingly, the total length of the light diffusing body 32 is approximately from 1.5 to 17 cm, preferably from 3 to 11 cm, although the ranges vary depending on the shape of a target tissue for treatment.

A diameter of the light diffusing body 32 is from 0.1 to 1.0 mm, preferably from 0.13 to 0.5 mm.

The present embodiment described an example where only a single light diffusing body 32 was provided. In this case, irradiation and light receiving may be performed by using a single light diffusing body, by a switching between a time zone for irradiation and a time zone for light receiving along the time axis. However, the example is not limited thereto, but may comprise two bodies of: a light diffusing body for irradiation to be used in irradiating a target site for treatment with a light from a light source; and a light diffusing body for monitoring to be used in receiving a returned fluorescence and measuring information on level of a lesion.

Since the light diffusing body for irradiation and the light diffusing body for monitoring are adjacent to each other, a contact or a friction therebetween can cause a concern that an interference (crosstalk) of signals leaked from the respective light diffusing body generates a signal deterioration. Therefore, the light diffusing body for irradiation and the light diffusing body for monitoring may be adhered to each other with a transparent adhesive, or a transparent spacer may be disposed between the bodies.

The shape memory wire 33 is inserted through the lumen 23 for shape memory wire.

The shape memory wire 33 is a wire that is formed of a nickel titanium alloy having an approximately circular cross section and has a shape memory property. As the shape memory wire 33, it is also possible to use an iron-based shape memory alloy such as an iron-manganese-silicon alloy, or a bimetal which is formed of two metal sheets joined together, each metal having a different coefficient of thermal expansion.

The shape memory wire 33 is formed so as to maintain the shape in which one end of the curved spiral shape of about 1.1 convolutions is bent at an angle of 90° or more, so that the shape of the catheter tube 20 as in FIG. 1 and FIG. 3 is maintained under a condition without an external stress.

The end portion in the distal side of the shape memory wire 33 is fixed to a tip electrode 29T, and the end portion in the proximal side is fixed to the tubular portion 10.

The shape memory wire 33 has an elasticity, and in a condition under a pressure from outside, is transformable in response to the pressure. Therefore, when the catheter tube 20 is in a blood vessel, pressure from a side wall of the blood vessel causes the wire to take a shape such as a gentle curve conforming to a shape of the blood vessel.

A gap is provided between the inner face of the lumen 23 for shape memory wire and the shape memory wire 33, and the inner face of the lumen 23 for shape memory wire forms an interface between material of in the catheter tube 20 and gas phase inside the lumen 23 for shape memory wire. Incidentally, by a movement of the catheter tube 20 inside a living body, the shape memory wire 33 is sometimes brought into contact with the inner face of the lumen 23 for shape memory wire. However, since the shape memory wire 33 and the inner face of the lumen 23 for shape memory wire are not constantly in contact or in close contact with each other, the inner face of the lumen 23 for shape memory wire forms an interface between the material of the catheter tube 20 and the gas phase inside the lumen 23.

A tension wire 34, linked to a publicly known control handle (not illustrated) for bending the end portion in the distal side of the catheter tube 20 by a drawing operation of an operator using the control handle at hand, is inserted through the lumen 24 for tension wire.

A gap is provided between the inner face of the lumen 24 for tension wire and the tension wire 34, and the inner face of the lumen 24 for tension wire forms an interface between material of the catheter tube 20 and gas phase inside the lumen 24 for tension wire. In this connection, by a movement of the catheter tube 20 inside a living body, the tension wire 34 is sometimes brought into contact with the inner face of the lumen 24 for tension wire. However, since the tension wire 34 and the inner face of the lumen 24 for tension wire are not constantly in contact or in close contact with each other, the inner face of the lumen 24 for tension wire forms an interface between the material of the catheter tube 20 and the gas phase inside the lumen 24 for tension wire.

In this connection, it is also possible to configure the catheter tube without providing the tension wire 34, and without inserting a solid substance into the lumen 24 for tension wire. By configuring in this manner, no solid substance abuts on the inner face of the lumen 24 for tension wire, and the inner face of the lumen 24 for tension wire forms an interface between material of the catheter tube 20 and gas phase or liquid phase inside the lumen 24 for tension wire.

The electrode wires 35 and 36 are respectively inserted through the lumens 25 and 26 for electrode wire. The electrode wires 35 and 36 are formed of a lead wire used in common electrode catheters, and each of the ten electrode wires 35 and 36 is connected by caulking to any of the nine ring electrodes 29R and a tip electrode 29T formed on the outer circumference of the catheter tube 20, by a publicly known method.

Between the inner faces of the lumens 25 and 26 for electrode wire and the electrode wires 35 and 36, gaps are provided, and the inner faces of the lumens 25 and 26 for electrode wire form interfaces between material of the catheter tube 20 and gas phase inside the lumens 25 or 26 for electrode wire. In this connection, by a movement of the catheter tube 20 inside a living body, the electrode wires 35 and 36 are sometimes brought into contact with the inner faces of the lumens 25 or 26 for electrode wire. However, since the electrode wires 35 and 36 and the inner faces of the lumens 25 and 26 for electrode wire are not constantly in contact or in close contact with each other, the inner faces of the lumens 25 and 26 for electrode wire form interfaces between the gas phases in the catheter tube 20 and inside the lumens 25 or 26 for electrode wire.

In this connection, it is also possible to configure the catheter tube without providing the electrode wires 35 and 36, and without inserting anything into the lumens 25 and 26 for electrode wire. By configuring in this manner, no solid substance abuts on the inner faces of the lumens 25 and 26 for electrode wire, and thus, the inner faces of the lumens 25 and 26 for electrode wire constantly form interfaces between material of the catheter tube 20 and gas phase or liquid phase inside the lumens 25 or 26 for electrode wire.

The inner faces of the lumen 23 for shape memory wire, the lumen 24 for tension wire, and the lumens 25 and 26 for electrode wire form interfaces between material of the catheter tube 20 and gas phases inside the each lumen 23 to 26. Since refractive index of the material of the catheter tube 20 and gas phase inside the each lumen 23 to 26 have different refractive indexes, the wall face of each of the lumens 23 to 26 forms an interface of the refractive indexes and functions as a reflection body for reflecting a light diffused from the light diffusing body 32.

Since the inner wall of each of the lumens 23 to 26 functions in this way as a reflection body for reflecting a light diffused from the light diffusing body 32, it is not necessary that the catheter tube 20 of the present embodiment comprise a mirror or a prism for the purpose of adjusting a radiation efficiency or a radiation distribution. Thus, the catheter tube 20 may be mirrorless and prismless, to allow configuring a catheter tube 20 having a high flexibility and a favorable maneuverability in a living body.

In this connection, each of the lumens 23 to 26 may be filled with a liquid such as saline, and in this case, the inner face of each of the lumens 23 to 26 forms an interface with the liquid phase.

The catheter tube 20 of the present embodiment comprises the lumen 22 for light diffusing body, the lumen 23 for shape memory wire, and the lumen 24 for tension wire arranged such that the centers of each lumens 22 to 24 are on the same straight line in the radius direction of the loop-like shaped portion 20c, in a cross section cut along the radius direction of the loop-like shaped portion 20c and in a direction perpendicular to the extending direction of the catheter tube 20, as shown in FIG. 3. The pair of lumens 25 and 26 for electrode wire is arranged in the side of the outer circumference of loop-like shaped portion 20c relative to the center of the lumen 23 for shape memory wire, line symmetrically about a straight line in the radius direction of the loop-like shaped portion 20c, in the both side sandwiching the lumen 23 for shape memory wire.

In the present embodiment, a diameter of the lumen 24 for tension wire is, for example, 350 μm. The centers of the lumens 25 and 26 for electrode wire are at positions 300 μm away from the center axis of the catheter tube 20 to the side of the lumen 22 for light diffusing body, in a direction of a straight line connecting each center of the lumen 22 for light diffusing body, the lumen 23 for shape memory wire, and the lumen 24 for tension wire. At the same time, the centers of the lumens 25 and 26 for electrode wire are at positions 400 μm away from the center axis of the catheter tube 20, to the side of the outer circumference, in a direction perpendicular to the straight line connecting each center of the lumen 22 for light diffusing body, the lumen 23 for shape memory wire, and the lumen 24 for tension wire.

In FIG. 3, provided that the radius direction of the loop-like shaped portion 20c is y-axis, and a direction perpendicular to the y-axis and to the extending direction of the catheter tube 20 is x-axis, an angle θ from the y-axis may be referred to as a wide angle, according to the present embodiment.

The catheter tube 20 of the present embodiment may be used for insulating an abnormal electrical conduction at an inlet portion of a pulmonary vein shown in FIG. 2, in a treatment of tachyarrhythmia.

Inlet portion of a pulmonary vein, together with superior vena cava, is one of treatment targets of atrial fibrillation which occupies the largest number of patient of tachyarrhythmias.

Pulmonary vein includes four pulmonary veins which are left superior pulmonary vein (LSPV), left inferior pulmonary vein (LIPV), right superior pulmonary vein (RSPV), and right inferior pulmonary vein (RIPV), as shown in FIG. 2. When these tissues are irradiated with light, the light irradiation is performed not with bringing the outer side of the loop-like shaped portion 20c of the catheter tube 20 into contact with the tissue, but in such a state that the loop-like shaped portion 20c is pressed against the left atrial wall. Due to a ridge R shown in FIG. 2 present in the right side of LSPV and LIPV, the loop-like shaped portion 20c cannot be pressed well in some examples, resulting in an unstable contact angle of the catheter tube with a tissue. In light of the above, in an inlet portion of a pulmonary vein, it is necessary that the angle θ shown in FIG. 2 be increased; namely, a light irradiation to a wide range is required.

Thus, the catheter tube 20 of the present embodiment capable of wide angle irradiation can be preferably used in insulating an abnormal electrical conduction at an inlet portion of a pulmonary vein.

### Embodiment 2: Catheter Tube 20′ Used in Cancer Treatment, Etc.

Hereinbelow, a catheter tube 20′ according to Embodiment 2 is described.

The catheter tube 20′ of the present embodiment is a catheter tube for cancer treatments by photosensitization reaction. Differently from the catheter tube 20 of Embodiment 1 used in treatments of tachyarrhythmias, the catheter tube 20′ is formed not into a looped shape, but into a linear shape, and may comprise an electrode, as needed. The catheter tube 20′ is capable of wide angle irradiation, and preferably used in treatments of cancers, particularly, for example, peripheral lung cancer.

In a treatment of cancer, the catheter tube 20′ of the present embodiment is used with being inserted into a channel of an endoscope (not illustrated) so as to be endoscopically inserted into a living body.

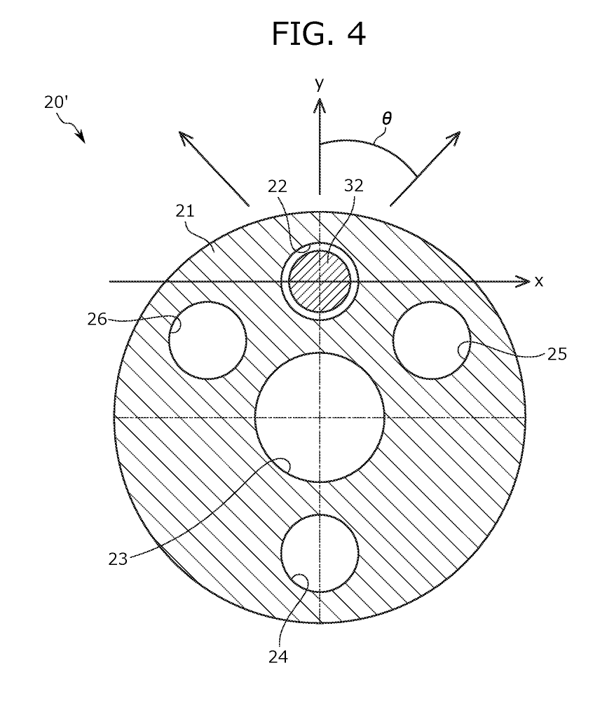

The catheter tube 20′ of the present embodiment is a modification of the catheter tube 20 according to Embodiment 1, and differs from the catheter tube 20 in that the lumen 23 for shape memory wire, the lumen 24 for tension wire, and the lumens 25 and 26 for electrode wire are not penetrated through by a wire, as shown in FIG. 4.

The catheter tube 20′ is formed into a linear shape, for not comprising the shape memory wire 33. The catheter tube 20′ differs from FIG. 1 also in the point that no electrode is provided around the catheter tube 20′.

In the catheter tube 20′ of the present embodiment, a wall face of each of the lumens 23 to 26 forms an interface of refractive indexes, and functions as a reflection body for reflecting a light diffused from the light diffusing body 32. The catheter tube 20′ of the present embodiment does not need to comprise a mirror or a prism for the purpose of adjusting a radiation efficiency or a radiation distribution. Thus, the catheter tube 20′ may be mirrorless and prismless, and accordingly, has a high flexibility and a favorable maneuverability inside a living body and in a channel of an endoscope.

The catheter tube 20′ of the present embodiment can be used, for example, in treatments of peripheral lung cancer, for being capable of wide angle radiation. The catheter tube 20′ of the present embodiment is preferably used in peripheral lung cancers, since a peripheral lung cancer can have an extensive area of target site for treatment, and at the same time, has low risk of occurrence of a stricture after a treatment due to a photosensitization reaction by a diffusing light irradiation.

### Embodiment 3: Catheter Tube 30 Used in Treatment of Arrhythmia

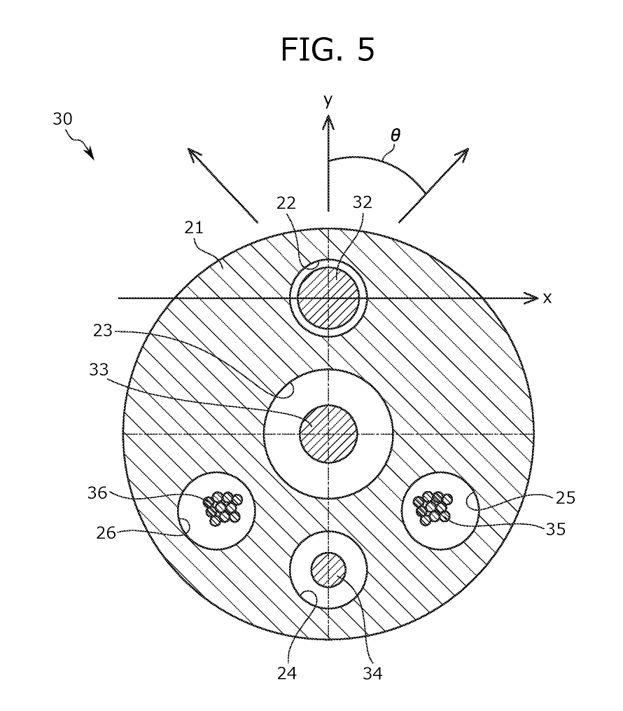

FIG. 5 shows catheter tube 30 according to Embodiment 3.

The catheter tube 30 of the present embodiment is, for example, a catheter tube which is capable of narrow angle radiation, and used in insulating an abnormal electrical conduction in a superior vena cava, in a treatment of tachyarrhythmia.

Exterior of the laser catheter 1 is the same as in Embodiment 1, and the catheter tube 30 forms a loop-like shaped portion 20c as shown in FIG. 1.

FIG. 5 shows a cross sectional view of the catheter tube 30 at a part corresponding to the A-A line cross section of FIG. 1.

The catheter tube 30 has the same structure with the catheter tube 20, except that the pair of lumens 25 and 26 for electrode wire is arranged in the side of the inner circumference, namely, in the side of the center C of the loop-like shaped portion 20c, relative to the center of the lumen 23 for shape memory wire, line symmetrically about a straight line in the radius direction of the loop-like shaped portion 20c, in the both sides sandwiching the lumen 23 for shape memory wire.

In the present embodiment, a diameter of the lumen 24 for tension wire is, for example, 350 μm. The centers of the lumens 25 and 26 for electrode wire are at positions 300 μm away from the center axis of the catheter tube 20 to the opposite side from the lumen 22 for light diffusing body, in a direction of a straight line connecting each center of the lumen 22 for light diffusing body, the lumen 23 for shape memory wire, and the lumen 24 for tension wire. At the same time, the centers of the lumens 25 and 26 for electrode wire are at positions 400 μm away from the center axis of the catheter tube 20, to the side of the outer circumference, in a direction perpendicular to the straight line connecting each center of the lumen 22 for light diffusing body, the lumen 23 for shape memory wire, and the lumen 24 for tension wire.

In FIG. 5, provided that the radius direction of the loop-like shaped portion 20c is y-axis, and a direction perpendicular to the y-axis and to the extending direction of the catheter tube 30 is x-axis, an angle θ from the y-axis may be referred to as a narrow angle, according to the present embodiment.

The catheter tube 30 of the present embodiment may be used in insulating an abnormal electrical conduction in a superior vena cava, in a treatment of tachyarrhythmia.

Since a light irradiation of superior vena cava is performed inside a blood vessel, it is possible to perform a treatment in a state that the outer side of the loop-like shaped portion 20c is in contact with a tissue. In order not to damage a sinoatrial node which is an origin of an endocardial stimulus conduction system in the vicinity of a target site for treatment, it is necessary to decrease the angle θ, namely, a light irradiation to local area is required.

Thus, the catheter tube 30 of the present embodiment capable of narrow angle radiation can be preferably used in insulating an abnormal electrical conduction in a superior vena cava.

### Embodiment 4: Catheter Tube 30′ Used in Cancer Treatment, Etc.

Hereinbelow, a catheter tube 30′ according to Embodiment 4 is described.

The catheter tube 30′ of the present embodiment is a catheter tube for cancer treatments by photosensitization reaction. Differently from the catheter tube 30 of Embodiment 3 used in treatments of tachyarrhythmias, the catheter tube 30′ is formed not into a loop-like shape, but into a linear shape, and not provided with an electrode. The catheter tube 30′ is capable of narrow angle radiation, and used, for example, in treatments of central lung cancers, malignant brain tumors, particularly, brainstem tumors, residual tumor after surgical operation, etc.

In a treatment of cancer, the catheter tube 30′ of the present embodiment is used with being inserted into a channel of an endoscope (not illustrated) so as to be endoscopically inserted into a living body.

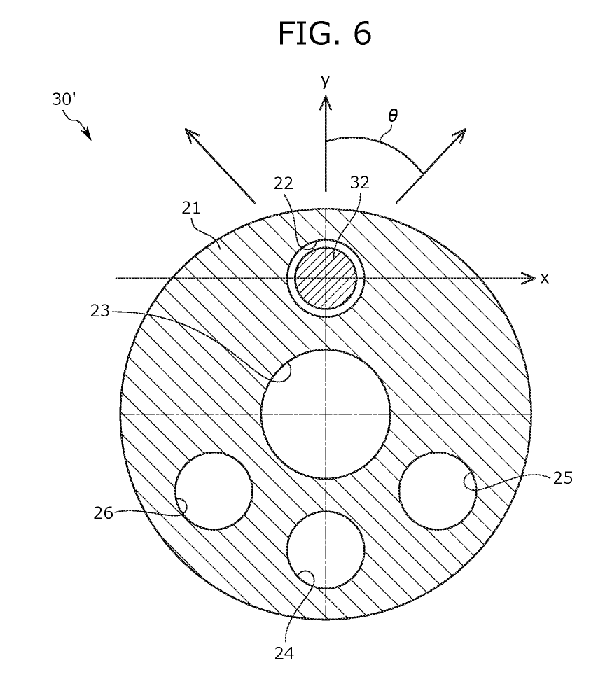

The catheter tube 30′ of the present embodiment is a modification of the catheter tube 30 according to Embodiment 3, and differs from the catheter tube 30 in that the lumen 23 for shape memory wire, the lumen 24 for tension wire, and the lumens 25 and 26 for electrode wire are not penetrated through by a wire, as shown in FIG. 6.

The catheter tube 30′ is formed not into a spiral shape of FIG. 1, but into a linear shape, for not comprising the shape memory wire 33. The catheter tube 30′ differs from FIG. 1 also in the point that no electrode is provided around the catheter tube 30′.

In the catheter tube 30′ of the present embodiment, a wall face of each of the lumens 23 to 26 forms an interface of refractive indexes, and functions as a reflection body for reflecting a light diffused from the light diffusing body 32. The catheter tube 30′ of the present embodiment does not need to comprise a mirror or a prism for the purpose of adjusting a radiation efficiency or a radiation distribution. Thus, the catheter tube 30′ may be mirrorless and prismless, allowing configuring a catheter tube 30′ having a high flexibility and a favorable maneuverability inside a living body.

Use of the catheter tube 30′ of the present embodiment in malignant brain tumors is described.

In recent years, with regard to malignant brain tumors, photodynamic treatments have come to be conducted on residual tumor cells which could not be removed by a surgical treatment. Specifically, when a brain tumor is extracted by an operation, a hole which is called extraction cavity is left. Around the extraction cavity, wet tumor cells are left in a normal brain. In conventional photodynamic treatments, inside of the extraction cavity was irradiated with a laser light, by using an irradiation unit provided outside a body. In malignant brain tumor tissues, a speed of metabolizing an agent is slow as compared to that in a normal tissue. As a result, a photosensitive agent accumulates only to tumor tissues, allowing only tumor tissues to be selectively treated, and thus, survival rate can be improved by the additional effect to a conventional multidisciplinary treatment.

However, by such a current light irradiation device, it is not possible to provide a sufficient radiation exposure dose to a wall of an extraction cavity. Moreover, since there are many important blood vessels or brain tissues around a tumor tissue in a brain, there are tumors in an inapproachable part.

On the other hand, the catheter tube 30′ of the present embodiment is capable of securing a sufficient irradiation amount to an extraction cavity, allowing a site which is currently inapproachable to be endoscopically irradiated with light.

For example, the catheter tube 30′ of the present embodiment may be made of a fluoro resin heat-shrinkable tube (FEP), having an outer diameter of about 800 μm.

### Other Embodiments

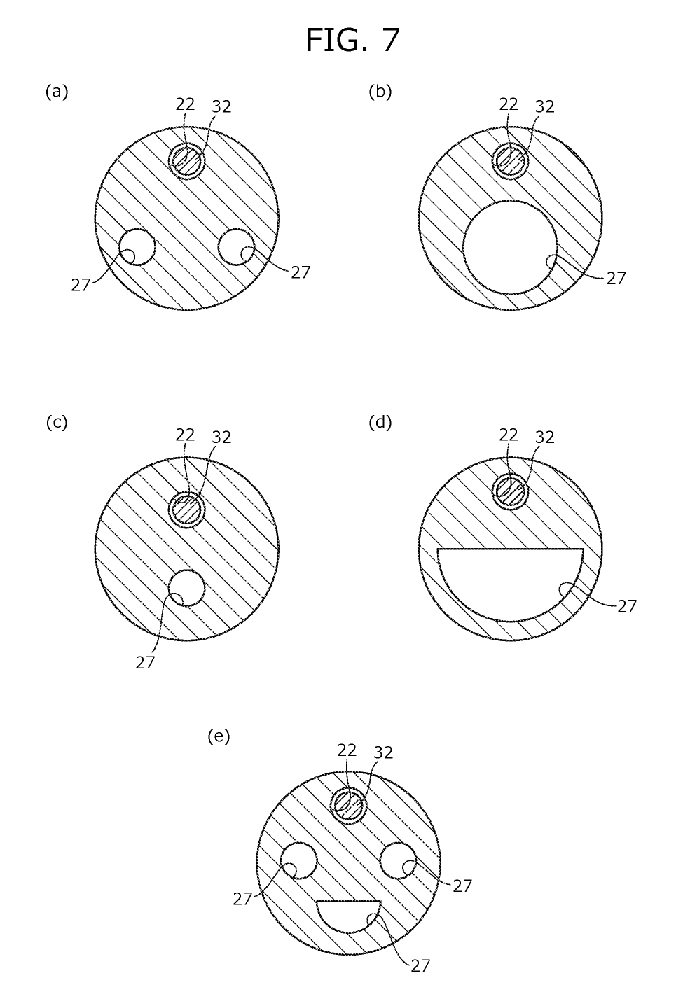

FIGS. 7(a) to (e) show structures of modifications of the catheter tube of the present invention other than Embodiments 1 to 4, in transections thereof. The catheter tubes of FIGS. 7(a) to (e) are catheter tubes for photodynamic treatments of cancer, which are inserted into a channel of an endoscope (not illustrated) so as to be endoscopically inserted into a living body.

The catheter tubes of FIGS. 7(a) to (e) all comprise the lumen 22 for light diffusing body and one or more lumens 27 for reflection having nothing inserted inside, and are composed in a line symmetrical manner about a straight line connecting the center of the lumen 22 for light diffusing body and the center of the catheter tube.

Other structures of the catheter tubes of FIGS. 7(a) to (e) are the same as those of the catheter tubes 20′ and 30′ according to Embodiments 2 and 4.

The catheter tubes of FIGS. 7(a) to (e) each control light radiation distribution by reflecting a light radiated from the light diffusing body 32 on an interface of a wall face of the lumen 27 for reflection.

### Examples

Hereinbelow, verification experiments and simulations of the radiation properties of the catheter tube according to the present invention are described.

In the experiments and simulations below, radiation distributions from catheter tubes to be models were measured, and calculation models explanatory thereof were produced. By using the produced models, each of the catheter tubes according to the embodiments of the present invention having desired light radiation efficiency and light radiation distribution was designed.

<Measurement of Radiation Distribution from 4 and 5 Lumen-Catheter Tubes 50a and 50b>

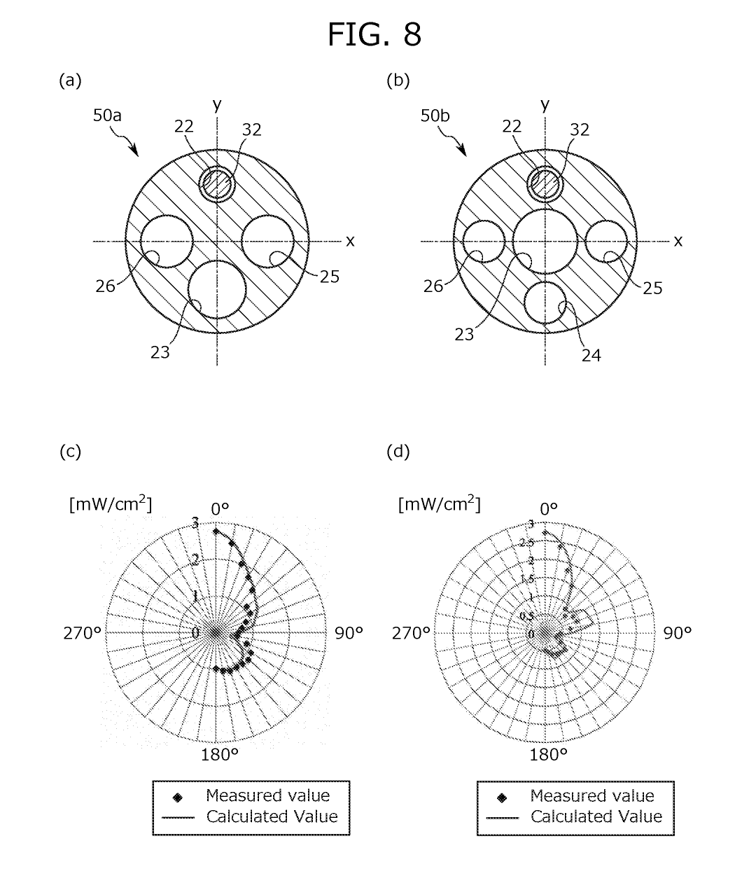

In the present measurement, radiation distributions were measured by using the 4 lumen- and 5 lumen-catheter tubes 50a and 50b of FIGS. 8(a) and (b) as the models, for the purpose of confirming radiation distributions of the catheter tubes according to the embodiments of the present invention.

Production of System for Measuring Radiation Distribution from Catheter Tube

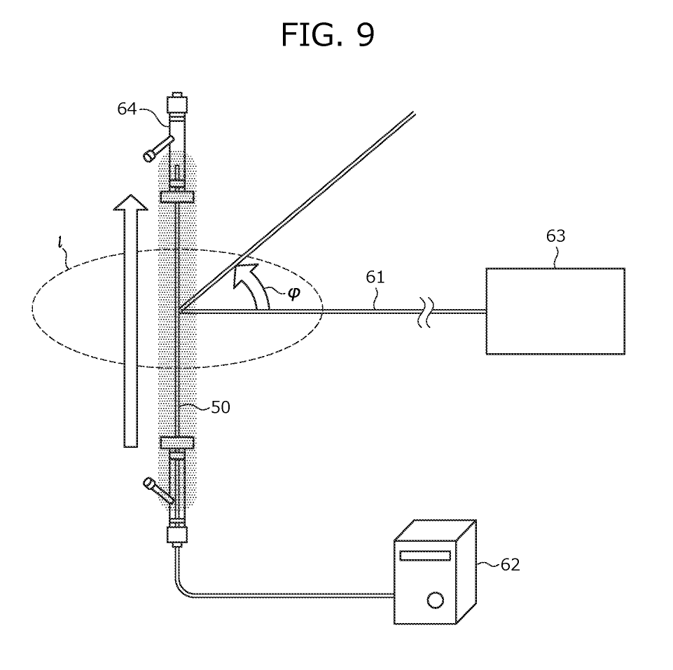

In order to measure radiation distributions from a catheter tube internally comprising a light diffusing body, an experimental system as shown in FIG. 9 was produced. As optical fiber 61 for measurement, an optical fiber having a core diameter: 200 μm and NA (number of aperture): 0.43 was used. An automatic stage (SHOT-GS, SIGMAKOKI Co., LTD.) (not illustrated) was used to move and rotate the optical fiber 61 for measurement. Resolutions in the longitudinal axis direction and in the periphery direction were 0.1 mm and 0.5°, respectively. As laser light source 62, a semiconductor laser (Rouge-LD, Cyber laser) with a center wavelength of 663 nm, having a lasing wavelength region in the Q band of a photosensitive agent, talaporfin sodium was used. For a measurement of light intensity, Silicon photodiode 63 (OP-2, VIS, Coherent) was used. To evaluate accuracy of the produced experimental system, Cylindrical Light Diffuser (Medlight SA) which was already developed into a product was used as the catheter tube 50 which internally comprised a light diffusing body capable of providing a stable light irradiation, and radiation intensity distribution at 20 mm from the bottom was measured for 10 times. Percentage of standard deviation to an average value was less than 5%, showing that an experimental system capable of measuring a radiation intensity distribution from the catheter tube 50 with a very high accuracy was produced.

Measurement of Radiation Distribution from 4 and 5 Lumen-Catheter Tubes 50a and 50b

By using the experimental system, radiation distributions from 4 lumen- and 5 lumen-catheter tubes 50a and b were measured. FIGS. 8(a) and (b) shows schematic cross-sectional views of the 4 lumen- and 5 lumen-catheter tubes 50a and b which were used in the measurement.

In the 4 lumen-catheter tube 50a, diameter of the lumen 22 for light diffusing body and diameters of the lumens 25 and 26 for electrode wire were 350 μm, and diameter of the lumen 23 for shape memory wire was 450 μm.

In the 5 lumen-catheter tube 50b, diameter of the lumen 22 for light diffusing body, diameters of the lumens 25 and 26 for electrode wire, and diameter of the lumen 24 for tension wire were 350 μm, and diameter of the lumen 23 for shape memory wire was 450 μm.

In the measurement, the 4 lumen- and 5 lumen-catheter tubes 50a and b had a light diffusing body 32 having diameter of 250 μm installed inside, and the other lumens 23 to 26 had no metal wire installed inside, and had air inside. In the used 4 lumen- and 5 lumen-catheter tubes 50a and b, inner walls of the each lumens 22 to 26 were not processed.

Firstly, examined was whether or not an eccentricity of the light diffusing body 32 (diameter 250 μm) inside the lumen 22 (diameter 350 μm) for light diffusing body influences a radiation distribution in the 4 lumen-catheter tube 50a and 5 lumen-catheter tube 50b.

In this examination, the light diffusing body 32 was inserted into a single lumen-tube (diameter 350 μm) (not illustrated) and radiation distribution in the periphery direction at position 35 mm from the bottom was measured. Surface radiation intensity of the light diffusing body 32 was set to 30 mW/cm². An average value and standard deviation when the measurement was repeated for 30 times were calculated.

The average value of the radiation intensity was 12.25 mW/cm², the standard deviation was 0.53 mW/cm², and the percentage of standard deviation to an average value was 4.3%. Accordingly, it was judged that an eccentricity of the light diffusing body 32 inside the lumen 22 for light diffusing body does not influence a radiation intensity distribution.

Subsequently, radiation distributions in near-field in the periphery direction from the 4 lumen- and 5 lumen-multiple hole catheter tubes 50a and b were measured. By using a medical device having a gasket structure (not illustrated), both ends of the 4 lumen-catheter tube 50a and the 5 lumen-catheter tube 50b were fixed. Then, the optical fiber 61 for measurement (core diameter: 200 μm, NA (number of aperture): 0.43) was moved and rotated by an automatic stage (SHOT-GS, SIGMAKOKI Co., LTD.) (not illustrated) in the longitudinal axis direction (direction I) and in a periphery direction (direction θ) of the catheter tubes 50a and 50b with a resolution of 0.1 mm and 0.5°, respectively, to thereby measure radiation intensities from the catheter tubes 50a and 50b in the periphery direction at the position 35 mm from the bottom. As the light diffusing body 32 provided inside the lumen 22 for light diffusing body of the catheter tubes 50a and 50b, a light diffusing body having an emission length of 70 mm was used, and output was set to 0.5 mW/cm. The measurement was conducted for 10 times, using the 4 lumen- and 5 lumen-catheter tubes 50a and 50b, and an average value was calculated. Since the standard deviation of the 10 times measurements was less than 5% of the average value, it was judged that the produced experimental system was capable of measuring a radiation distribution from a catheter tube with a high accuracy.

Results of the measurements of radiation distributions in the near-field in the periphery direction from the 4 lumen- and 5 lumen-catheter tubes 50a and 50b are shown in FIGS. 8(c) and (d) with dots.

### <Calculation of Radiation Distribution of 4 and 5 Lumen-Catheter Tubes 50a and 5013>

Ray Tracing Simulation by Monte-Carlo Method

Next, radiation distributions from transparent catheter tube with multiple holes were calculated by a ray tracing simulation according to Monte-Carlo Method, using a conventional simulation software. The Monte-Carlo Method is a method of stochastically predicting phenomenon such as irradiation, absorption and diffusion of light, by generating uniform random numbers in a simulation, which is capable of determining a traveling direction and optical path of light waves. It is possible to calculate a behavior of a light within a substance having a complicated cross sectional structure, such as a transparent catheter tube with multiple holes.

The Monte-Carlo Method considers a light flux radiated from a light source as an assembly of many particles, and randomly analyzes a step length and a step direction of a photon (S. L. Jacques et al., Optical-thermal response of laser-irradiated tissue, pp. 72-83, 1995; L. Wang et al., Computer Methods and Programs in Biomedicine, vol. 47, pp. 131-146, 1995).

Step length L of a photon is a distance traveled by a photon after a collision with a particle until another collision occurs, which is represented as follows, provided that R (0<R<1) is a random number, an absorption coefficient is μₐ, and a scattering coefficient is μₛ.

$$L = - \frac{ln R}{{μ}_{s} + {μ}_{a}} \qquad [ Mathematical Formula 1 ]$$

A photon will have an intensity attenuation exponentially as represented by the following formula, using a propagation distance z (integration of L) and an effectual attenuation coefficient μ_{eff}, and will extinct when reached a specific value.

I(z)=I₀exp(−μ_{eff}z) [Mathematical Formula 2]

A new step length of a photon is in accordance with a phase function P (θ). In handling a complex multiple scattering, Henyey-Greenstein function (M. J. C. V. Gemert et al., IEEE Transactions on biomedical engineering, vol. 36, pp. 1146-1154, 1989.) represented by the following formula is used in many cases.

$$f ( {s}^{i n} , {s}^{out} ) = P ( θ ) = \frac{1}{4 π} [ \frac{1 - {g}^{2}}{{( 1 + {g}^{2} - 2 g cos θ )}^{3 / 2}} ] π {r}^{2} \qquad [ Mathematical Formula 3 ]$$

g is called as anisotropic parameter, being an average of phase functions that represent scattering angles, and is represented by the formula as below which integrates phase functions with all angles (Sachio YAMADA, Medical imaging technology, vol. 10, pp. 490-496, 1992).

g=<cos θ>=∫_{4π}p(cos θ)·cos θdω [Mathematical Formula 4]

g may take a value of from 1 (absolute forward scattering) to −1 (absolute backward scattering), and value 0 represents an absolute isotropic scattering. Generally, in a living tissue, value of from 0.80 to 0.97 which represent almost forward scattering is obtained. When a strong forward scattering is occurring in a substance, a state similar to an isotropic scattering in appearance is observed, due to a scattering repeated several times in a tissue. Thus, an equivalent scattering coefficient μₛ′ for a case of regarding a scattering as an isotropic scattering is represented as follows (Minoru OHARA, et al., Practical Laser Engineering, CORONA PUBLISHING CO., LTD., pp. 167-177, 1998).

μₛ′=(1−g)μₛ [Mathematical Formula 5]

In the present simulation, radiation distribution from a catheter tube was calculated, by using a ray tracing simulation by the Monte-Carlo Method. An absorption coefficient μₐ and an equivalent scattering coefficient μₛ′ of a substance are required in order to calculate a behavior of a light in a catheter tube by using a ray tracing simulation by the Monte-Carlo Method.

Measurement of Transmissivity and Reflectivity of Tube Material

Thus, in order to calculate radiation distributions from a catheter tube, an absorption coefficient μₐ and an equivalent scattering coefficient μₛ′ of a catheter tube at wavelength of 663 nm were measured.

In order to calculate the absorption coefficient μₐ and the equivalent scattering coefficient μₛ′ at wave length of 663 nm of Pebax (registered trademark) (55D) which is a material for the catheter tube, five pieces of Pebax (registered trademark) (55D) bulk material, having a thickness of 1 mm were prepared, and individual transmissivity and reflectivity were measured with a spectrophotometer (UV-3600, SHIMADZU) having an integrating-sphere (ISR-3100, SHIMADZU).

Calculation of Optical Constant of Tube by Inverse Adding Doubling Method

On the basis of the obtained transmissivity and reflectivity of the Pedax (registered trademark) (55D), absorption coefficient μₐ and equivalent scattering coefficient μₛ′ of the tube were calculated by Inverse Adding Doubling Method (S. A. Prahl et al., Applied optics, vol. 32, pp. 559-568, 1993).

Inverse Adding Doubling Method is a method of calculating absorption coefficient μₐ and equivalent scattering coefficient μₛ′, based on Adding Doubling Method.

Process of Adding Doubling Method is described. A thin uniform single layer having a known absorption coefficient μₐ and equivalent scattering coefficient μₛ′ is produced, and transmissivity and reflectivity thereof are calculated. Transmissivity and reflectivity in a case where an identical layer is superimposed on this base (adding), and in a case where a different layer is superimposed on this base (doubling) were calculated. This process was repeated until a desired thickness is obtained, and a transmissivity and a reflectivity of a desired final specimen are calculated.

The Inverse Adding Doubling Method is an inverse problem of this Adding Doubling Method, where, first, an absorption coefficient μₐ and an equivalent scattering coefficient μₛ′ are presumed; a transmissivity and a reflectivity are calculated by the Adding Doubling Method, on the basis of the presumed absorption coefficient μₐ and equivalent scattering coefficient μₛ′; the calculated values and measured values are compared; and the process is repeated until the values agree.

Average values of the calculated absorption coefficient μₐ and of the equivalent scattering coefficient μₛ′ of the five specimens were 1.27×10⁻² mm⁻¹, and 1.27×10⁻¹ mm⁻¹, respectively. Standard deviations were 2.24×10⁻³ mm⁻¹, and 4.73×10⁻³ mm⁻¹, which were 1.76% and 3.73% of the average values, respectively. Since the standard deviations of the absorption coefficient μₐ and the equivalent scattering coefficient μₛ′ were much smaller than the average values, an absorption coefficient μₐ and an equivalent scattering coefficient μₛ′ to be input to the calculation model were the respective average values of 1.27×10⁻² mm⁻¹ and 1.27×10⁻¹ mm⁻¹.

Calculation of Radiation Distribution of 4 Lumen- and 5 Lumen-Catheter Tubes 50a, 50b

By using the measured absorption coefficient μₐ and equivalent scattering coefficient μₛ′ of the Pedax (registered trademark) (55D), a model capable of calculating radiation distributions from 4 lumen- and 5 lumen-catheter tubes 50a and b, having the same dimension with those used in the measurement was produced. The calculation model in the present simulation is conceived to be applied also to a calculation of radiation intensity distribution inside a living body. Therefore, a ray tracing simulator according to the Monte-Carlo Method was used. Solid works (Dassault Systems Solidworks) was used as a 3D-CAD software, and Optis works (Optis) was used as a ray tracing software. The OPTIS works is a software capable of conducting an optical analysis by inputting optical constants such as absorption coefficient, equivalent scattering coefficient, or anisotropic parameter to a model data designed on Solid works which is a 3D-CAD software capable of assembling parts from planning.

In the present simulator, it is possible to set a number of rays radiated from a light source. Number of rays corresponds to number of calculation in the Monte-Carlo Method, and a larger number of rays allow a calculation to be performed with a higher accuracy. In the present simulation, number of rays was set as large as one million, in order to cover complicated light propagations, such as absorption, scattering inside a catheter tube, or reflection on an interface of each lumen. In order to compare values with measured values, an absorption body capable of imitating the optical fiber of NA: 0.43 used in the measurement was installed around a light receiving face. Output and wavelength from a light diffusing body was set to 0.5 mW/cm and 663 nm, as in the measurement.

Radiation intensity distributions from the 4-lumen and 5-lumen catheter tubes 50a and b were calculated by using the produced calculation model, to obtain the solid lines in FIGS. 8(c) and (d). The radiation intensity at 0° was the maximum, and the radiation intensity at 90° was the minimum value which was smaller than the radiation intensity at 180°, similarly as in the measurement.

Comparison Between the Result of Measurement and Result of Calculation of Radiation Distribution

Next, the results of measurement and the results of calculation of radiation distributions from the 4-lumen and 5-lumen catheter tubes 50a and b were compared to verify adequacy of the produced calculation model.

Table 1 and Table 2 show measured values and calculated values of radiation intensities in directions of 0, 30, 60, 90, 120, 150, and 180°, in distributions of radiations from the 4-lumen and 5-lumen catheter tubes 50a and b.

TABLE 1
Radiation intensity at surface of 4 lumen-catheter tube 50a

| Angle [°] | 0 | 30 | 60 | 90 | 120 | 150 | 180 |
|---|---|---|---|---|---|---|---|
| Measured value | 2.76 | 1.75 | 1.07 | 0.60 | 1.09 | 1.10 | 0.98 |
| [mW/cm²] | | | | | | | |
| Calculated value | 2.78 | 1.79 | 1.29 | 0.61 | 0.78 | 1.08 | 0.96 |
| [mW/cm²] | | | | | | | |
| Deviation [%] | 0.78 | 2.55 | 20.7 | 1.29 | 28.6 | 1.44 | 2.18 |

TABLE 2
Radiation intensity at surface of 5 lumen-catheter tube 50b

| Angle [°] | 0 | 30 | 60 | 90 | 120 | 150 | 180 |
|---|---|---|---|---|---|---|---|
| Measured value | 2.73 | 1.48 | 0.90 | 0.43 | 0.50 | 0.65 | 0.48 |
| [mW/cm²] | | | | | | | |
| Calculated value | 2.77 | 1.47 | 1.21 | 0.77 | 0.57 | 0.75 | 0.44 |
| [mW/cm²] | | | | | | | |
| Deviation [%] | 1.43 | 0.81 | 34.6 | 78.1 | 14.2 | 15.0 | 9.34 |

As in Table 1, in the 4 lumen-catheter tube 50a, deviations between the measured values and the calculated values at 60° and 120° exceeded 20% which was the allowable measurement deviation of a medical device, while deviations in the other angles were less than 3%. Similarly, as in Table 2, in the 5 lumen-catheter tube 50b, deviations between the measured values and the calculated values at 60° and 90° exceeded 20% which was the allowable measurement deviation of a medical device, while deviations in the other angles were less than 15%. To sum up the above, concerning the measured values and the calculated values of surface radiation intensities of the 4 lumen- and 5 lumen-catheter tubes 50a and b, the radiation distribution shapes agreed in general, except for some portions.

In order to compare measured values and calculated values of radiation angles, angles at which radiation intensity on the surface of a catheter tube becomes 30%, 50%, and 70% of the maximum value were obtained. Table 3 and Table 4 show radiation angles of 4 lumen- and 5 lumen-catheter tubes 50a and b.

TABLE 3
Radiation angle from 4 lumen-catheter tube 50a

| | Radiation angle at | Radiation angle at | Radiation angle at |
|---|---|---|---|
| | which radiation | which radiation | which radiation |
| | intensity becomes | intensity becomes | intensity becomes |
| | 30% of the | 50% of the | 70% of the |
| | maximum | maximum | maximum |
| Measured value | 79.0° | 45.5° | 24.1° |
| Calculated | 78.4° | 50.0° | 27.5° |
| value | | | |
| Deviation [%] | 0.76 | 9.89 | 14.1 |

TABLE 4
Radiation angle from 5 lumen-catheter tube 50b

| | Radiation angle at | Radiation angle at | Radiation angle at |
|---|---|---|---|
| | which radiation | which radiation | which radiation |
| | intensity becomes | intensity becomes | intensity becomes |
| | 30% of the | 50% of the | 70% of the |
| | maximum | maximum | maximum |
| Measured value | 41.9° | 31.3° | 20.0° |
| Calculated | 41.1° | 32.0° | 21.6° |
| value | | | |
| Deviation [%] | 1.91 | 2.24 | 8.00 |

From Table 3 and Table 4, deviations between the measured values and the calculated values at the defined angles of radiation from the 4 lumen- and 5 lumen-catheter tubes 50a and b were less than 20% at radiation angles of all conditions. In light of the above, it was judged that the measured values and the calculated values of the radiation angle had a high consistency.

In order to compare the measured values and the calculated values of the radiation efficiency of the 4 lumen- and 5 lumen-catheter tubes 50a and b, the plots in the graphs shown in FIGS. 8(c) and (d) were linked with straight lines which were integrated from 0° to 180°. Integrated value of the measured radiation distribution values of the 4 lumen-catheter tube 50a was 70.8 mW/cm²; integrated value of the calculated values was 71.1 mW/cm²; and deviation between the measured values and the calculated values was 0.42%. Integrated value of the measured radiation distribution values of the 5 lumen-catheter tube 50b was 51.6 mW/cm²; integrated value of the calculated values was 60.0 mW/cm²; and deviation between the measured values and the calculated values was 14%. In both of the 4 lumen- and 5 lumen-catheter tubes 50a and b, the deviation between the measured values and the calculated values of radiation efficiency was less than 20%, and it was judged that there is a high consistency.

In light of the above, it is considered that the measured values and the calculated values of the radiation distribution shape, the surface radiation intensity, radiation angle, and radiation efficiency substantially agreed. The imitation tube used in the calculation model this time comprises an inorganic substance, and considered to have little variation in optical constant. Therefore, in the present simulation, an absorption coefficient μₐ and an equivalent scattering coefficient μₛ′ input without adjustment of optical constants were taken. That is, the produced calculation model was judged to be adequate. The following Table 5 collects input parameters of the calculation models produced in the present simulation.

TABLE 5
State of produced calculation model and input parameter

| | | | Tube | | | |
|---|---|---|---|---|---|---|
| Output from | | Tube | equivalent | Reflectivity | Number | Number |
| light diffusing | | absorption | scattering | of lumen | of ray | of ray |
| body | Wavelength | coefficient | coefficient | interface | (calculation) | (Ray-trace) |
| 500 mW | 663 nm | 1.27 × 10⁻² mm⁻¹ | 1.27 × 10⁻¹ mm⁻¹ | 10% | 1,000,000 | 200 |

| Scattering coefficient input to tube | Tube refractive index | Plating refractive index |
|---|---|---|
| Henyey-Greenstein function | 1.49 | 95% |

Study of Arrangement of Lumens 25 and 26 for Electrode Wire

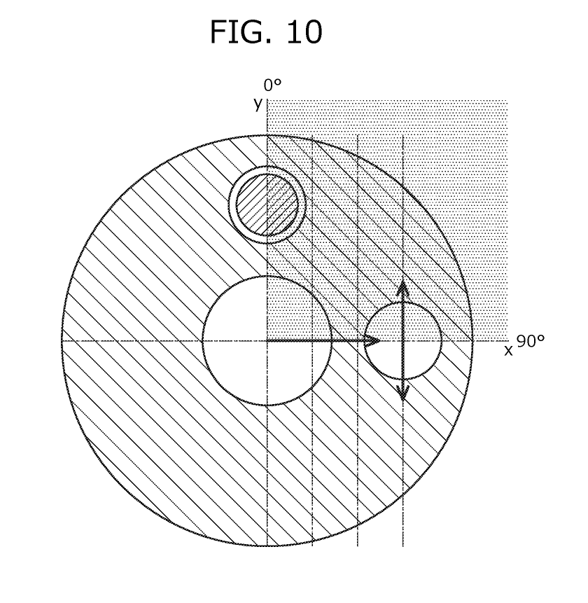

Next, a light irradiation property, when a position of the lumen 25 for electrode wire of Embodiments 1 to 4 and FIG. 8(b) was moved, was investigated. A position of the light diffusing body 32 was fixed to a position of 0.1 mm tube wall thickness, and the lumen 25 for electrode wire was moved in the direction x, and in direction y of FIG. 10 at intervals of 0.1 mm. The direction y is a linear direction toward the center axis of the lumen for light diffusing body from the center axis of the catheter tube, on the vertical section of the catheter tube; and the direction x is a linear direction toward the outer circumference direction of the catheter tube from the center axis of the catheter tube, which is perpendicular to the direction y, on the vertical section of the catheter tube. 0.1 mm is the minimum position resolution of an insertion hole in an actual tube development.

Ranges of changing were set as: when x: 0 mm, y: in a range of from −0.4 to 0.1 mm; when x: 0.1 mm, y: in a range of from −0.4 to 0.1 mm; when x: 0.2 mm, y: in a range of from −0.4 to 0.2 mm; when x: 0.3 mm, y: in a range of from −0.4 to 0.3 mm; and when x: 0.4 mm, y: −0.3 to 0.3 mm.

At this time, the inner wall of the lumen 25 for electrode wire was supposed to have a reflectivity of 95%, on a supposition of a gold plating application. The gold plating is applied onto the inner wall of the lumen 25 for electrode wire, since it prevents the electrode wire 35 from a temperature rise to increase a reflectivity, and since there is a possibility that wire absorptions or reflection spectra vary depending on a quenching temperature at the time of forming the metal wire. The light radiation properties which were investigated for relationship with the arrangement of the lumen 25 for electrode wire were three properties which were radiation angle, radiation efficiency, and maximum radiation intensity on a surface of the tube. The radiation angle was defined to be an angle where a radiation intensity becomes 50% of the maximum radiation intensity.

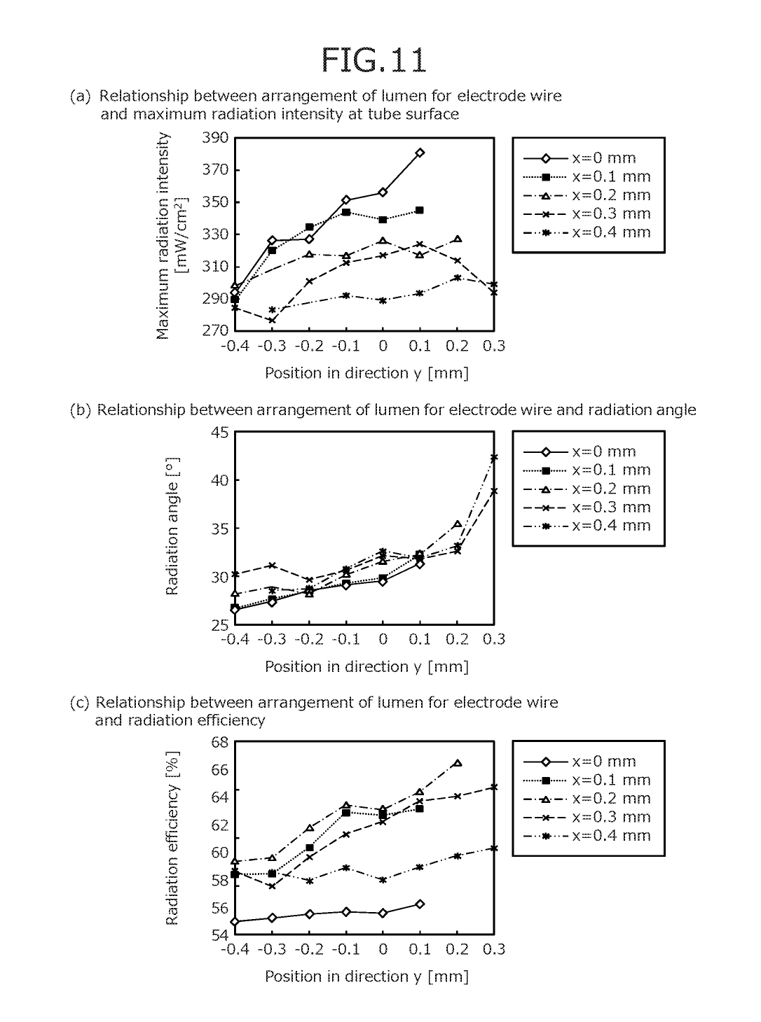

FIG. 11 shows relationships between the positions of the lumen 25 for electrode wire obtained by using the produced calculation model and each of the light radiation properties.

As shown in FIG. 11(a), the smaller the direction x position of the lumen 25 for electrode wire was, the larger the maximum radiation intensity became, and the maximum radiation intensity exhibited an increased tendency with an increase of position in direction y, except for the cases where the position in direction x was 0.3 or 0.4 mm. The radiation intensity was the maximum at a position 0.1 mm in the direction y when the position in direction x was 0.3 mm; and at a position 0.2 mm in the direction y when the position in direction x was 0.4 mm.

As shown in FIG. 11(b), the radiation angle did not depend on a position in direction x, and exhibited an increased tendency with an increase of a position in direction y. The radiation efficiency similarly exhibited an increased tendency with an increase of a position in direction y. With regard to the radiation angle, as the lumen 25 for electrode wire moves away from the light diffusing body 32, an incidence angle of a light toward the lumen wall becomes smaller, and a reflection angle also becomes smaller, and accordingly, a radiation angle of a light radiated from a catheter tube is considered to become smaller as well.

In FIG. 11(c), the results are considered to be reasonable in respect to radiation intensity and transmission rate, because a mean free path of a light inside a catheter tube is decreased by placing another lumen in the vicinity of the light diffusing body 32. Although a device capable of light irradiation to a local area and at a high efficiency is generally needed, it was presumed, in the design of the present catheter tube, that radiation efficiency was in antinomy with radiation angle, when a light irradiation of a superior vena cava (SVCI) was supposed. In other words, it is considered that, by an arrangement of lumens which increases radiation efficiency, a radiation angle is also increased.

From results of the above study, the catheter tubes 20′ and 30′ of FIG. 4 and FIG. 6 were produced within the calculation models, and radiation distributions were calculated.

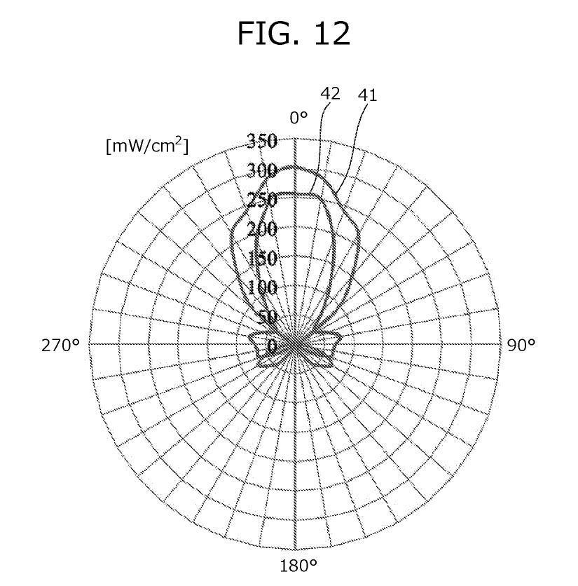

FIG. 12 shows the results thereof. The radiation distribution 41 shows a radiation distribution of the catheter tube 20′ of FIG. 4, and the radiation distribution 42 shows a radiation distribution of the catheter tube 30′ of FIG. 6.

A catheter tube having a low radiation efficiency cannot give an energy necessary to a treatment, and moreover, a lost energy is converted into a heat to cause a rise of temperature of a tube, and therefore, a design of radiation efficiency is important. However, it is considered that a sufficient treatment energy can be obtained by increasing Input. In light of the above, in the embodiments of the present invention, the catheter tubes 20, 20′, 30, and 30′ of FIGS. 3 to 6 were designed giving priority to design of radiation distribution.

As in FIG. 12, the catheter tube 20′ of FIG. 4 exhibited a radiation distribution of wide angle, and the catheter tube 30′ of FIG. 6 exhibited a radiation distribution of narrow angle, from which it was found that there is a difference in radiation distribution.

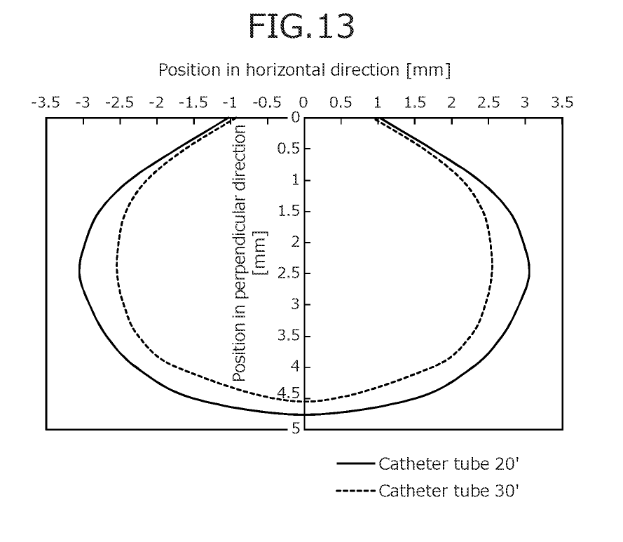

The catheter tubes 20′ and 30′ exhibited radiation intensities of 50% of the maximum radiation intensity, at angles of approximately 45° and 25°. It is considered that a radiation angle can be designed within this range. FIG. 13 shows a distribution inside tissue, when a myocardial tissue is irradiated with a light from the catheter tube 20′ or 30′ in a state in contact with the tissue perpendicularly. FIG. 13 shows a boundary at which a radiation intensity inside the tissue becomes 4.3 mW/cm², when Output from the light diffusing body 32 is 50 mW/cm. The light intensity of 4.3 mW/cm² is a threshold for radiation intensity necessary to a treatment within 10 minutes, which was obtained through an in vivo animal experiment (agent concentration: 20 pg/ml).

In this case, it is considered that a difference of about 1-2 mm occurs in a treatment width, as in the graph of FIG. 13. The above fact that a difference also occurs in distribution inside a tissue suggests that the design of the present radiation distribution is effective. As described above, in the design of the present catheter tube, a radiation angle and a radiation efficiency are in antinomy with each other. Therefore, a tube capable of irradiating a local area will have a low radiation efficiency. Radiation efficiencies of the catheter tubes 20′ and 30′ having the structures presumed at this time were calculated to be 74% and 61%, respectively.

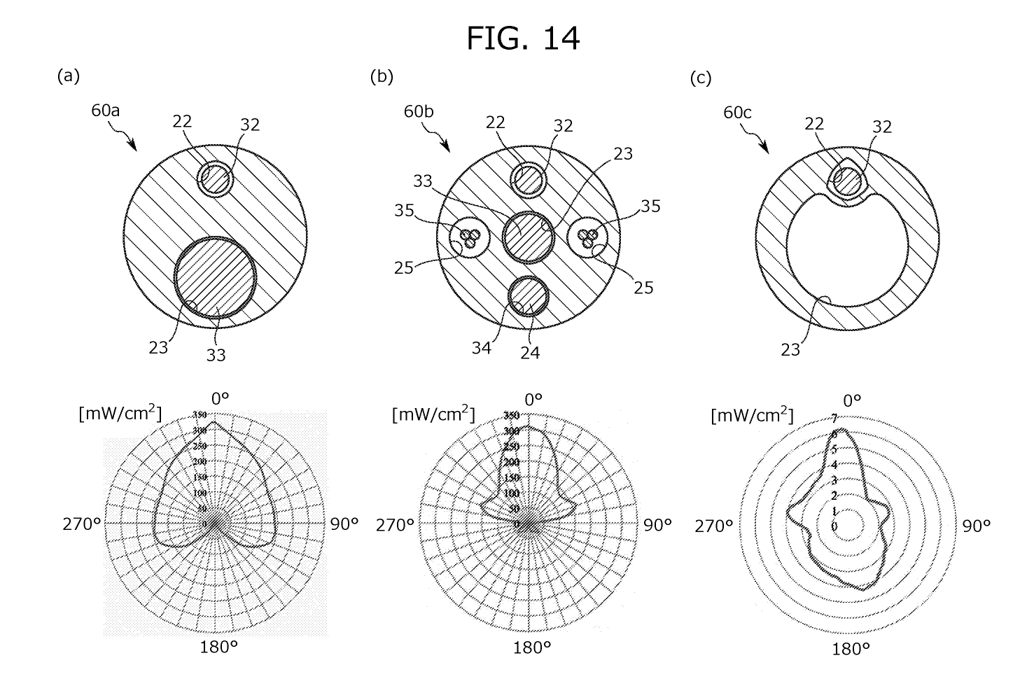

The catheter tubes according to the other examples of the present invention were measured for radiation distribution, and FIG. 14 shows a result of the measurements. FIG. 14 shows cross-sectional views of the catheter tubes 30 at a part corresponding to the A-A line section of FIG. 1 in the upper row, and radiation distributions of the catheter tubes 30 in the lower row.

The catheter tube 60a in FIG. 14(a) is a 2 lumen-catheter tube for brain surgery capable of being used in PDT to a malignant brain tumor, which was produced inside a ray tracing simulator. Outer diameter of the catheter tube was configured to be 1.00 mm, an outer diameter of the light diffusing body 32 was configured to be 0.25 mm, and outer diameter of the shape memory wire 33 was configured to be 0.43 mm. Diameter of lumen 22 for the light diffusing body was configured to be 0.35 mm, diameter of lumen 23 for the shape memory wire was configured to be 0.45 mm, and reflectivity of the outer side of the shape memory wire 33 was set to 95%, on a supposition of a gold plating application. Radiation intensity in the periphery direction of the catheter tube 60a was calculated, with setting number of rays to one million, and output from the light diffusing body 32 to 50 mW/cm. The lower diagram of FIG. 14(a) shows that it is a design well capable of irradiating one side, due to an influence of the shape memory wire 33. In order to configure the design to be capable of more local light irradiation, it is necessary to further increase the diameter of the shape memory wire 33. However, from a viewpoint of durability, the upper limit of the diameter is considered to be 0.45 mm in a tube having an outer diameter of 1 mm.

The catheter tube 60b in FIG. 14(b) is a 5 lumen-catheter tube internally comprising a variety of wires, having a tube diameter of 1.46 mm, in which outer diameter of light diffusing body 32 is configured to be 0.25 mm, outer diameters of electrode wires 35 and 36 are configured to be 0.1 mm, and outer diameter of shape memory wire 33 is configured to be 0.43 mm. Diameters of lumen 22 for the light diffusing body, lumens 25 and 26 for the electrode wires, and lumen 24 for a tension wire were configured to be 0.35 mm; diameter of lumen 23 for the shape memory wire was configured to be 0.45 mm; and the lumens 25 and 26 for the electrode wires were each internally provided with three electrode wires 35 and 36, respectively. Reflectivity of the outer side of the shape memory wire 33 was set to 95%, on a supposition of a gold plating application. Reflectivity of the electrode wires 35 and 36 was set to 60%, on a supposition that the wires comprise copper; and reflectivity of the tension wire 34 was set to 60%, on a supposition that the wire comprises stainless. Radiation intensity in the periphery direction of the catheter tube 60b was calculated, with setting number of rays to one million, and output from the light diffusing body to 50 mW/cm.

In the catheter tube 60c in FIG. 14(c), the lumen 22 for light diffusing body has a shape of kite (a shape in which diagonal lines intercross with each other, and sizes of two angles facing each other, formed with two edges which differ in length, are equal), and is formed with a shape having a roundish angles and edges. The lumen 23 for shape memory wire is provided in the side of center axis of the catheter tube 60c, relative to the lumen 22 for light diffusing body, as a lumen larger than the lumen 22 for light diffusing body. A wall face of the lumen 23 for shape memory wire in the side of the lumen 22 for light diffusing body is configured to be a shape formed along a wall face of the lumen 22 for light diffusing body, and the lumen 23 for shape memory wire is formed into a crescent shape. As shown in the lower diagram of FIG. 14(c), the catheter tube 60c radiates a light not only to the side of the light diffusing body 32, but also to the opposite side of the catheter tube 60c from the light diffusing body 32, and thus is capable of not one side radiation but both side radiation.

What is claimed is:

1. A catheter tube to be provided to a medical device which is inserted into a living body, and irradiates a target site for treatment or a target site for examination with a light to conduct a treatment or an examination of the site, the catheter tube comprising:
an insertion hole through which a light diffusing body is inserted, and one or more other holes which differ from the insertion hole, in a manner extending in a longitudinal direction of the catheter tube, wherein
at least a part of the catheter tube is made of an optically transparent material,
a space present inside each of the one or more other holes adjacent to an inner wall surface of each of the one or more other holes, and
the inner wall surface forms an optical interface between the optically transparent material and a gas phase or a liquid phase inside the space, which are different from each other in refractive index, to conjointly form a reflection surface for adjusting a radiation distribution of a light emitted by the light diffusing body.

2. The catheter tube according to claim 1 wherein the catheter tube does not comprise a reflection body that comprises a material different from the optically transparent material of the catheter tube.

3. The catheter tube according to claim 1, wherein the catheter tube does not comprise a mirror or a prism for adjusting radiation distribution made of a material different from the optically transparent material of the catheter tube.

4. The catheter tube according to claim 1 wherein the catheter is used in a treatment of arrhythmia and further comprises a loop-like shaped portion comprising a spiral shape of more than a single convolution under a condition without an external stress, wherein a tip portion comprising a bent portion which is bent at an end portion on a proximal side of the loop-like shaped portion.

5. The catheter tube according to claim 1, wherein
the catheter tube is a wide-angle radiation catheter tube for a treatment of tachyarrhythmia at an inlet portion of a pulmonary vein, or a treatment of peripheral lung cancer by a photosensitization reaction,
the other holes include first to fourth holes which are provided in a side of a center axis of the catheter tube rather than a center axis of the insertion hole,
the first hole is formed at a position substantially coaxial with the catheter tube,
the second hole is formed in an opposite side of the first hole from the insertion hole,
the catheter tube is formed such that the center axes of the insertion hole, the first hole, and the second hole are arranged in a straight line which passes through the center axis of the insertion hole and the center axis of the catheter tube, and
center axes of the third and the fourth holes are positioned between the center axis of the insertion hole and the center axis of the first hole, in a linear direction.

6. The catheter tube according to claim 1, wherein
the catheter tube is a narrow angle radiation catheter tube for a treatment of tachyarrhythmia in superior vena cava, or a treatment of central lung cancer, treatment of brainstem tumor or treatment of residual tumor after surgical operation by a photosensitization reaction,
the other holes include first to fourth holes which are provided in the side of a center axis of the catheter tube rather than a center axis of the insertion hole,
the first hole is formed at a position substantially coaxial with the catheter tube,
the second hole is formed in an opposite side of the first hole from the insertion hole,
the catheter tube is formed such that individual center axes of the insertion hole, the first hole, and the second hole are arranged in a straight line which passes through the center axis of the insertion hole and the center axis of the catheter tube, and
the third and the fourth holes are positioned between the center axis of the second hole and the center axis of the first hole, in a linear direction.

7. A method of conducting a treatment or an examination of cancer or arrhythmia in a target site for treatment or a target site for examination, the method comprising:
inserting a catheter tube provided to a medical device into a patient body; and
irradiating the target site with a light by using the catheter tube,
wherein the catheter tube comprises;
an insertion hole through which a light diffusing body is inserted, and one or more other holes which differ from the insertion hole, provided in a manner extending in a longitudinal direction of the catheter tube, wherein
at least a part of the catheter tube is made of an optically transparent material,
a space present inside each of the one or more other holes adjacent to an inner wall surface of each of the one or more other holes, and
the inner wall surface forms an optical interface between the optically transparent material and a gas phase or a liquid phase inside the space, which are different from each other in refractive index, to conjointly form a reflection surface for adjusting a radiation distribution of a light emitted by the light diffusing body.
